(19) United States
(12) Patent Application Publication
(10) Pub. No.: US 2004/0087978 A1
(43) Pub. Date: May 6, 2004

(54) Surgical fascia closure instrument, guide and method

(76) Inventors: Juan Manuel Velez, Ladera Ranch, CA (US); Laszlo Garamszegi, Mission Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/228,601

(22) Filed: Aug. 27, 2002

(51) Int. Cl. A61B 17/12

(52) U.S. Cl. 606/144

(57) ABSTRACT

A surgical instrument, a snap-on guide and method capable of being used for closure of fascia and other laparoscopic procedures that use large operative ports or trocars and is intended to help prevent post-operative hernia defects and is also used to stop acute bleeding from the abdominal wall. The instrument shaft with the needle tip is attached to a hand piece allowing the operator to control the retractable grasping surface. The needle guide directly applies onto the shaft of a trocar and it slides down to the level of the subcutaneous tissue helping the operator to place the needle. Once the suture is passed through the abdominal wall, it is released then the needle is re-inserted through the opposite “guide port”. The suture creates an intra-abdominal loop of suture outside the abdomen. Suture ends will be tied, allowing closure of the fascial defect within the abdominal wall.

### CROSS REFERENCE TO RELATED APPLICATIONS

Not Applicable

### STATMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of laparascopic surgery and more specifically to a surgical fascia closure instrument, guide and method.

1. Field of the Invention

The present invention relates to improvements in the procedure for suturing tissue during laparoscopic surgery. More particularly, the invention relates to a method of suturing which utilizes a modified laparoscopic grasper and a guide. An alternative embodiment of the laparoscopic grasper has an interchangeable shaft configuration.

2. Description of the Related Art

An endoscopic/laparoscopy procedure involves making small surgical incisions in a patient's body for the insertion of trocar tubes thereby creating access ports into the patient's body. Thereafter, various types of endoscopic/laparoscopic instruments are inserted through these access ports and the appropriate surgical procedures are carried out.

After the surgical procedure is performed, the trocar tubes are usually removed and the incisions sutured closed by using both a needle and grasper for penetrating the tissue and handling the suture. This procedure for closure is frequently a time-consuming procedure requiring the identification of the fascia and closure of each fascial site with suture from an external point.

The necessity for closing these port sites in laparoscopic surgery is critical since suturing the incisions improperly can lead to bowel herniation through the port sites as well as the possibility of omental trapping if the fascial sites are not properly closed. Incisional hernias have occurred in both laparoscopic-assisted vaginal hysterectomies and laparoscopic cholecystectomies as well as other advanced laparoscopic procedures.

Thus there is a need for an endoscopic/laparoscopic instrument, a guide and method which will significantly reduce the operating time and is better able to give the surgeon direct visualization of the fascial and peritoneal closing. Additionally, there is a need for a surgical instrument which allows the surgeon to control bleeding sites by rapidly putting sutures around blood vessels of the abdominal wall without the need to remove trocar tubes already in the wound.

U.S. Pat. No. 6,183,485 issued to Rodger D. Thomason on Feb. 6, 2001

This patent is directed to a suturing means and method using a laparoscopic surgical instrument and a guide. The laparoscopic surgical instrument comprises a modified laparoscopic grasper wherein forceps jaws at the tip are manipulated by means of handles extending from a tubular housing with an enclosed reciprocating actuating rod connected with the handlers. The laparoscopic surgical instrument of this patent has the tip of the forceps jaws modified to have either a knife-, chisel-, or cone-shaped tip when the jaws are in the closed position. The guide is used for directing the surgical instrument to accurately pierce the tissue and carry the suture to the predetermined area of the body. To place the guide within the body opening, the removal of the operating trocar tubes are necessary.

With the present invention, the suture guide is applied to the shaft of a trocar at the beginning of the surgery and slides down to the level of the subcutaneous tissue. Within the needle guide, there are two guide ports which orient the needle at selected angles to allow the needle to pass through the abdominal wall. Suturing can start immediately without frustration The surgeon simply passes the suture through the tissue, then picks up the suture for tying or passing through the tissue to create another stitch for wound closure. The present invention allows introduction of suture through large, 10 mm or greater operative ports or trocars.

Additionally, the technique for using the present invention is easily learned; and the several embodiments set forth herein generally reduce the time and frustration associated with intra-abdominal suturing. These advantages are enhanced by use of the guide disclosed herein.

### BRIEF SUMMARY OF THE INVENTION

It is an object of the present invention to provide a surgical instrument, a guide and a method for the closure of a surgical incision under direct camera vision of the surgeon.

A further object of the invention is to provide a laparoscopic instrument, a guide and a method that allows for the rapid control of bleeding vessels in the outer or the abdominal wall that may occur with the placement of laparoscopy trocars.

Still another object of the invention to provide a guide that will attach onto the trocar tube and eliminates trocar removal from the incision, therefore causes less trauma and allows the surgeon to start the facia closure procedure immidately.

Another object of the invention is to provide a laparoscopic instrument that easily disassembles by hands only for providing easy access to all the components for cleaning and sterilization prior to surgery.

It is another object of the invention to provide an improved guide to accurately and consistently restrain the position and angle of insertion of a laparoscopic instrument to provide for proper placement and retrieval of suture material at a predetermined location within the body.

Accordingly, it is an objective of the present invention to provide a method associated with an improved surgical instrument and an improved guide that better suits the needs of a surgeon when suturing closed a surgical incision.

These and other objects of and advantages of the present invention will be apparent from a review of the following specification and accompanying drawings.

Other objects and advantages of the present invention will become apparent from the following descriptions, taken in connection with the accompanying drawings, wherein, by way of illustration and example, an embodiment of the present invention is disclosed.

The present invention is directed to a suturing means and method using an improved probe guide and an improved laparoscopic surgical instrument which permits a surgeon to pass suture without trauma through tissue while retaining the function of grasping the suture.

The laparoscopic surgical instrument comprises a modified laparoscopic grasper wherein grasping surfaces close to the tip are manipulated by means of handles extending from a tubular housing with an enclosed reciprocating actuating rod connected with the handles.

The invention includes a snap on suture probe guide delivering guided access to appropriate tissue layers for suturing. In a preferred embodiment, the probe guide is attached on to the operating trocar which remains in the wound during a laparoscopic tissue closure procedure.

The drawings constitute a part of this specification and include exemplary embodiments to the invention, which may be embodied in various forms. It is to be understood that in some instances various aspects of the invention may be shown exaggerated or enlarged to facilitate an understanding of the invention.

### BRIEF DESCRIPTION OF DRAWINGS

The above, as well as other, advantages of the present invention will become readily apparent to those skilled in the art from the following detailed description of the preferred embodiments when considered in light of the accompanying drawings in which:

FIG. 1a. is a side elevational view of a laparoscopic instrument of the present invention

FIG. 1b. is an exploded side elevational view of the laparoscopic instrument of FIG. 1a.

FIG. 2. is a side elevational partial view of the tip of the laparoscopic instrument showing in closed condition

FIG. 3. is a side elevational sectional view of the tip of the laparoscopic instrument showing in closed condition

FIG. 4. is a side elevational partial view of the tip of the laparoscopic instrument showing in open condition

FIG. 5. is a side elevational broken view of the tip of the laparoscopic instrument showing in open condition

FIG. 6. is an isometric view of the tip of the laparoscopic instrument showing in open condition

FIG. 7. is a dimetric view of the tip of the laparoscopic instrument showing in open condition

FIG. 8a. is a diagrammatic sketch, partly broken away, of the surgical instrument in the closed position passing suture through tissue.

FIG. 8b. is a diagrammatic sketch, partly broken away, of the surgical instrument in the open position for dropping the suture.

FIG. 8c. is a diagrammatic sketch, partly broken away, of the surgical instrument in the closed position passing suture through tissue at the other side of the incision and picking up suture.

FIG. 8d. is a diagrammatic sketch, partly broken away, of the surgical instrument pulling suture through muscle fascia and peritoneum.

FIG. 8e. is a diagrammatic sketch, partly broken away, of the suture tied below the skin to complete closure.

FIG. 9a. is a side elevational view of an alternative embodiment to the laparoscopic instrument of the present invention.

FIG. 9b. is a perspective view of the forceps jaws in open and in closed position according to one embodiment of the invention.

FIG. 9c. is an isometric view of another alternative embodiment to the laparoscopic instrument of the present invention showing in closed position.

FIG. 9d. is a perspective detail view, of the forceps tip in closed position according to one embodiment of the invention.

FIG. 9e. is a perspective detail view, of the forceps tip in open position according to one embodiment of the invention.

FIG. 9f. is an isometric view of another alternative embodiment to the laparoscopic instrument of the present invention showing in open position.

FIG. 10a. is a diagrammatic sketch showing the guide of the present invention attached on the operating trocar within the wound to be closed receiving the tip of the surgical instrument received within a passageway carrying suture material.

FIG. 10b. is a diagrammatic sketch showing the guide with the surgical instrument releasing the suture material.

FIG. 10c. is a diagrammatic sketch showing the guide with the surgical instrument being received in an opposite and adjacent passageway of the guide retrieving the suture material.

FIG. 10d. is a diagrammatic sketch showing the guide with the surgical instrument pulling suture through muscle fascia and peritoneum.

FIG. 10e. is a diagrammatic sketch showing the operating trocar with the guide and the surgical instrument has been removed from the body and the loop of suture is ready for wound closure.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Referring now to the drawings wherein like reference numerals refer to like and corresponding parts throughout, the laparoscopic instrument is generally indicated by numeral 20.

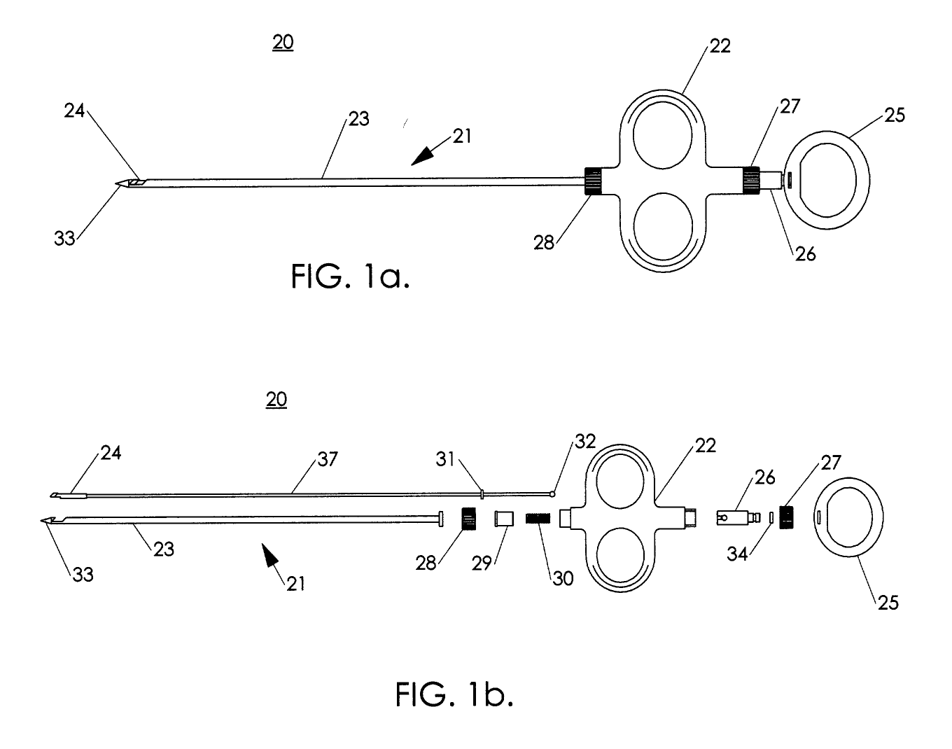

Referring now to FIGS. 1a and 1b , where grasping surface 35 is fixed and grasping surface 36 is moving back and forth when actuating rod 37 is reciprocated by a surgeon manipulating the instrument handle 22 and 23 providing a driving for driving grasping surface 36 to be in closed contact with fixed grasping surface 36 for carrying suture. Detachable means 21 comprise an elongated tube 23 concentrically sharing an axis with the actuating rod 37 having having grasping surfaces 35 and 36 engaged at a distal third of the outer shaft 23.

As shown in FIG. 1b , the laparoscopic instrument 20 can be easily disassembled for sterilization prior to surgery by separating handle 22 from detachable means 21 by loosening the knurled screws 28 on fixed handle housing 22,and other knurled screw 27 at thumbring 25 and unlatching connecting ball 32 from rotating piece 26 which thereby frees actuating rod 37 and tube 23 from handle housing 22. By loosening thumb screw 27, thumbring 25 can be disassembled from fixed handle housing 22 that allows for cleaning of the inside of the handle-housing area. When disassembled, the parts may be flushed, washed, and dried according to hospital procedures for stainless steel surgical instruments.

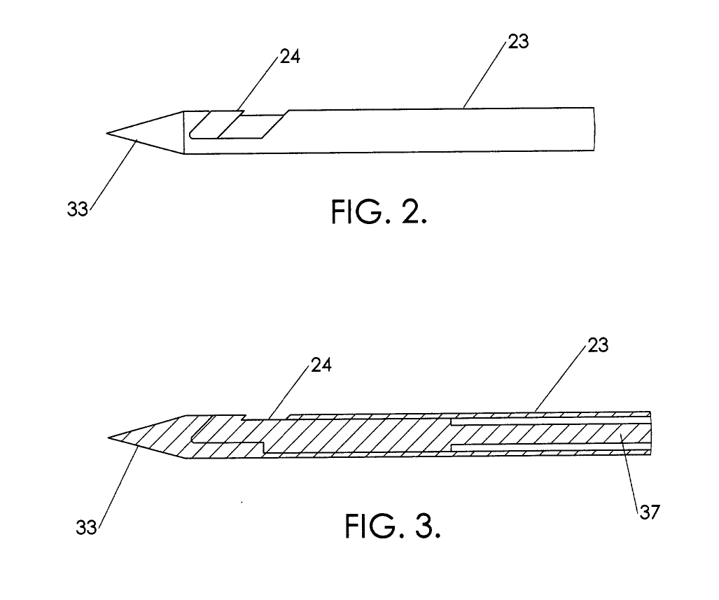

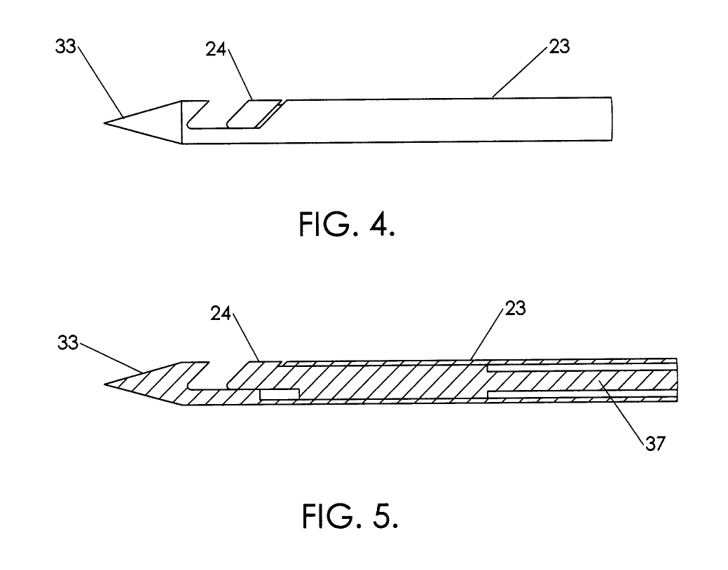

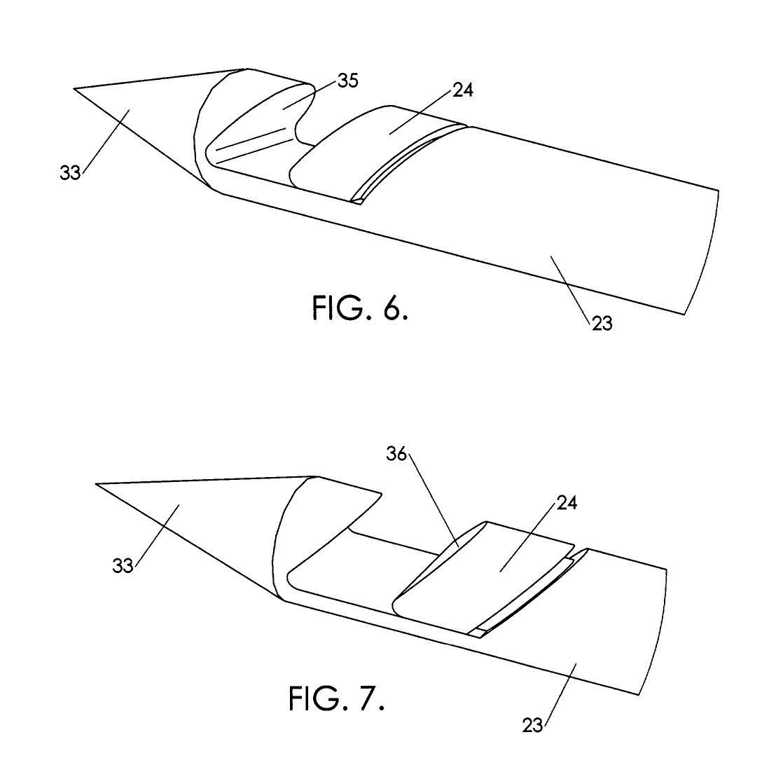

With the above-described arrangement, it will be seen that the surgeon is able to selectively operate the handle 22 and thumbring 23 to independently open and close the movable grasping surface 36 in relationship to fixed grasping surface 35 for grasping, carrying, or releasing suture during a laparoscopic operation. To open grasping surface 36, the surgeon moves movable thumbring 25 connected to rotating piece 26 forward toward the distal end of tube 23. As shown in FIGS. 2, 3, 4 and 5 the grasping surfaces 35 and 36 are separate from needle tip 33. The tip 33 operates as a sharp needle point that pierce through soft tissue while grasping surface 35 and 36 simultaneously grips and passes the suture. FIG. 4. shows the surgical instrument 20 in the open position. FIG. 5. shows a sectional side elevational view of the instrument and it also explains how the inner part 24 with the grasping surface 36 at the end moves inside the outer tube 23, FIGS. 2. and 3. shows the same instrument in a closed position.

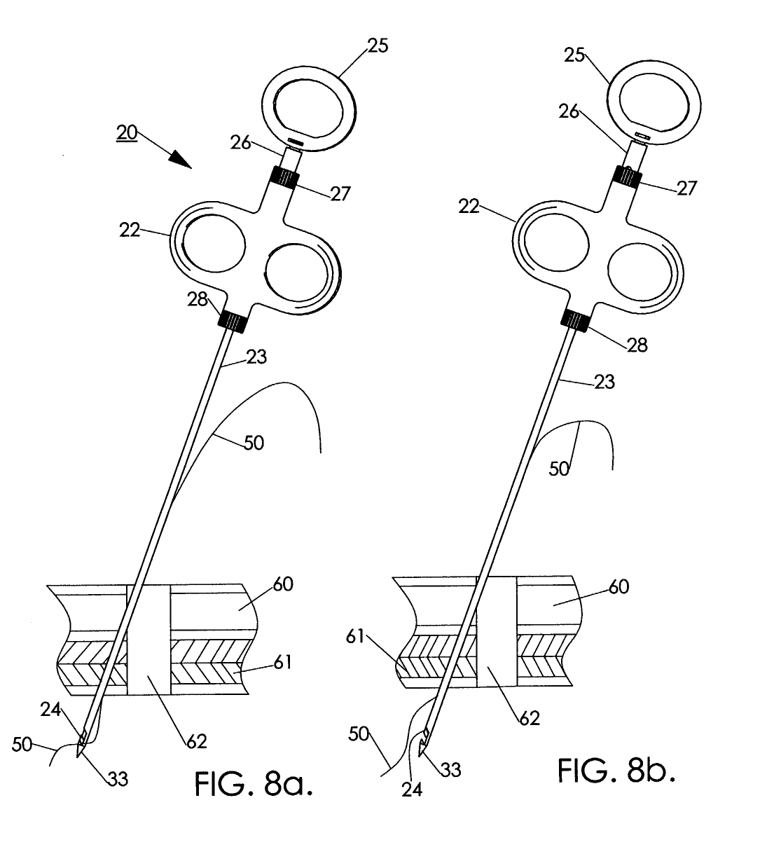

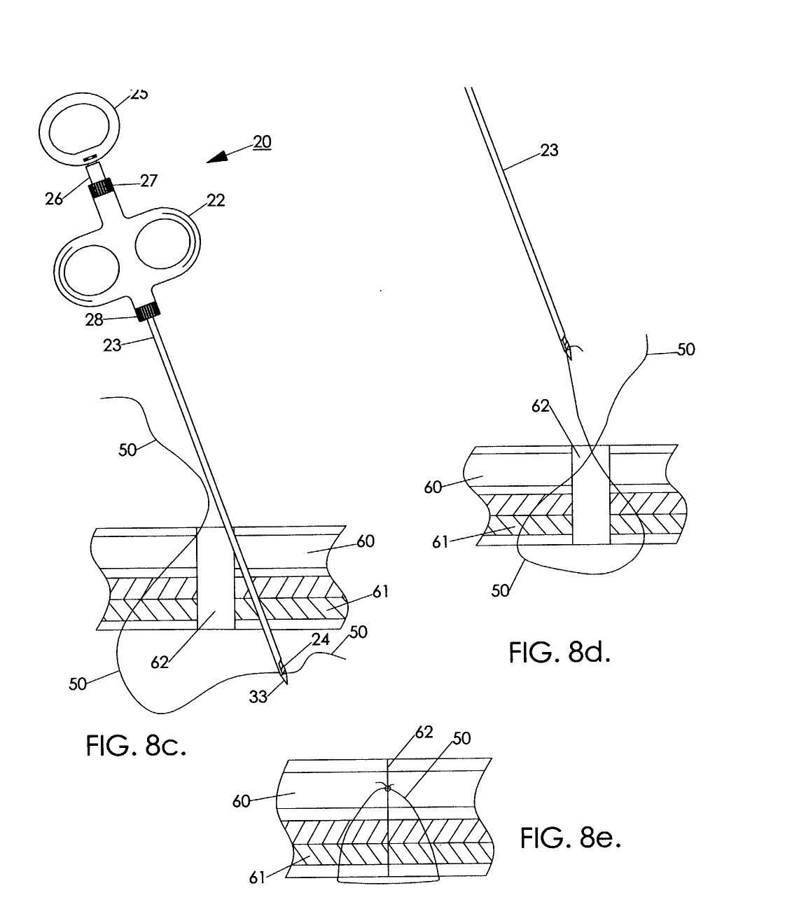

FIGS. 8a. through 8e. are diagrammatic representations of one example of using the method and laparoscopic instrument 20 of the present invention grasping and passing suture through soft tissue for closure of an incision 62. In FIG. 8a. the surgeon grasps the suture material 50 with grasping surfaces 35 and 36 and inserts instrument 20 carrying suture material 50 through the muscle fascia 60 and peritoneum 61 until the tip 33 and grasping surface 35 and 36 is seen through the peritoneum by direct camera vision. Subsequently, the surgeon releases the suture 50 by opening the moving grasping surface 36 located on the end of the moving inner part 24 and withdrawing the instrument 20 out of incision 62 as shown in FIG. 8b. In FIG. 8c. the surgeon then takes instrument 20 and inserts the tip 33 through the muscle fascia 60 and peritoneum 61 opposite the first point of insertion, grasping the suture 50 with gripping surface 35 and 36 and pulling the suture 50 carried and held by grasping surface 35 and 36 outside incision 62 as shown by FIG. 8d. whereupon suture 50 is tied below the skin to complete closure of incision 62 as shown by FIG. 8e.

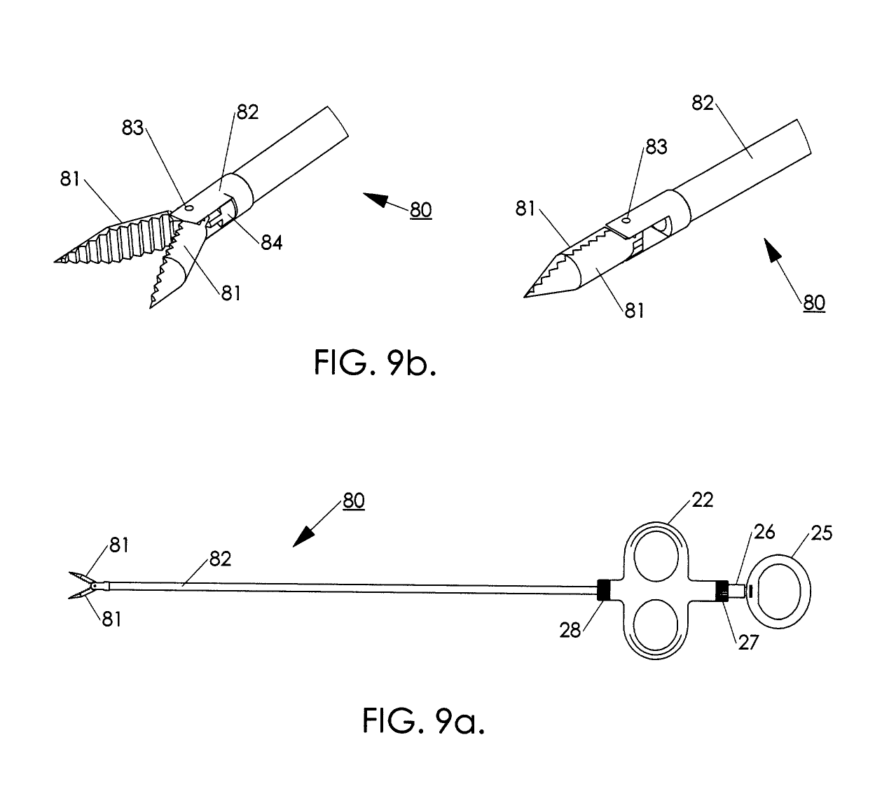

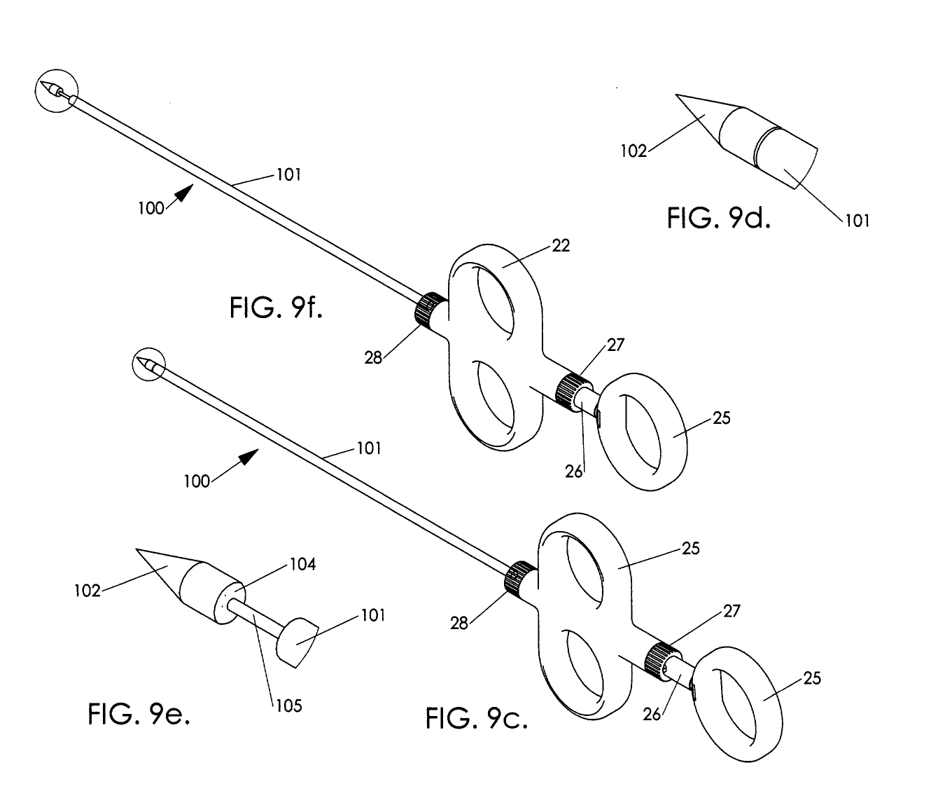

As shown in FIGS. 9a. through 9f., additional alternative embodiments of the present invention provide additional advantages for both specific and general applications.

FIG. 9b. shows an interchangeable grasping forcep shaft 80 with serrated jaws 81 forming a sharp, cone shaped needle tip in a fully closed position. The shaft 80 is connected to the handle 22 and thumbring 25 by tightening knurled screw 27 and 28. The interchangeable grasper shaft 80 has two identical jaws 81 at the tip. Both jaws 81 are are retractable by an inner actuating rod 84 allowing to pierce tissue and carry suture in the same time. A pin 83 serves as a pivot point for the moving jaws 81. The exceedingly sharp needle tip formed by the jaws 81 provides easy penetration of tissue layers. The option to be able to retract both jaws, allows the surgeon to manipulate the suture easier.

As shown on FIG. 9c, the novel configuration of the shaft 100 provides an alternative embodiment to that shown in FIGS. 1a. and 1b. The shaft 101 is fix and engaged to handle 22. An actuating rod 105 with a needle tip 102 at its end connected to handle 22 and thumbring 25, moves inside the outer tube defining a suture gripping area 106 (shown in FIG. 9d.) between grasping surface 104 and the paralell end of the outer tube 101. The advantage of this alternative is, there is no need to rotate the instrument for suture pick up because the gripping surface is concentrically located on the instrument shaft allowing suture grasping in 360 degrees.

FIG. 9c. shows the above alternative embodiment in a closed position and the detail view of the closed tip in FIG. 9d.

FIG. 9f. shows the above alternative embodiment in an open position and the detail view of the open tip in FIG. 9e.

Materials used to construct the devices set forth herein include surgical stainless steel and other alloys.

The present invention has been found to facilitate many camera-viewed laparoscopic procedures. By varying the diameter, length and curvature of the shaft, many procedures may be improved compared to previously-existing methods. Laparoscopic port closure and the identification and retraction of ureters during lympadenectomy also advantageously implement the present invention. The same is likewise true for retraction of kidneys and other structures during laparoscopic nephrectomy.

Intra-abdominal suturing, whether by closing of peritoneum or intra-abdominal knot-tying, has benefited from use of the present invention as has laparoscopic port closure (as for the urological uses listed above). In general surgery, the present invention has been found to be advantageously used with respect to laparoscopic port closures and temporary fixations of hernia mesh.

It is contemplated that many other surgical procedures will advantageously use the present inventive methods, guide and instruments as described herein.

These features and their advantages in use will be more particularly appreciated when reviewing the following method of the present invention used to pass suture through soft tissues during endoscopic/laparoscopic surgery for which the instrument 20 of this invention is provided. In application the surgical instrument 20 is to be grasped by a skilled laparoscopic surgeon and placed for closure of punctured vessels in the muscular surface or for closure of the fascia.

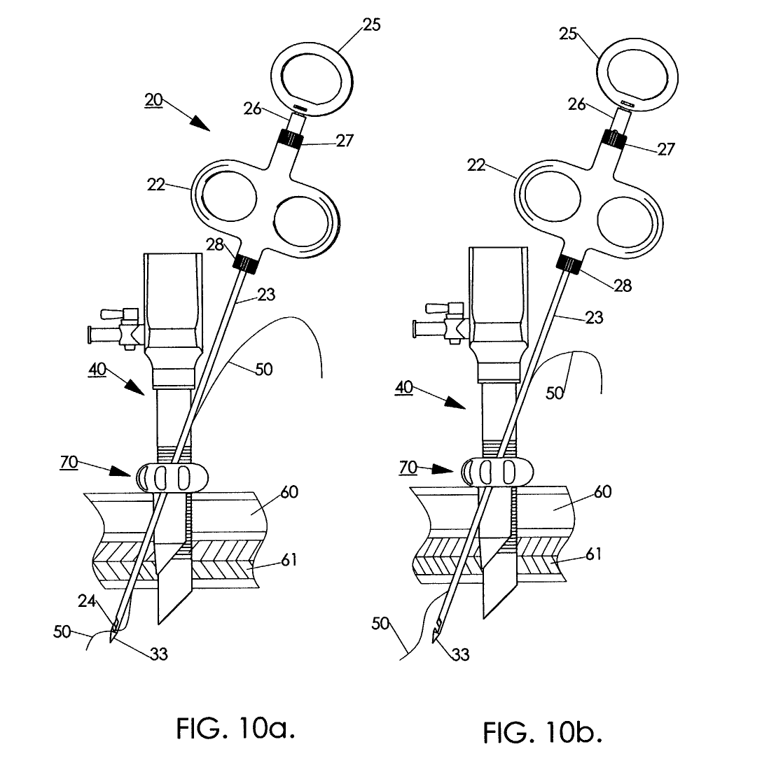

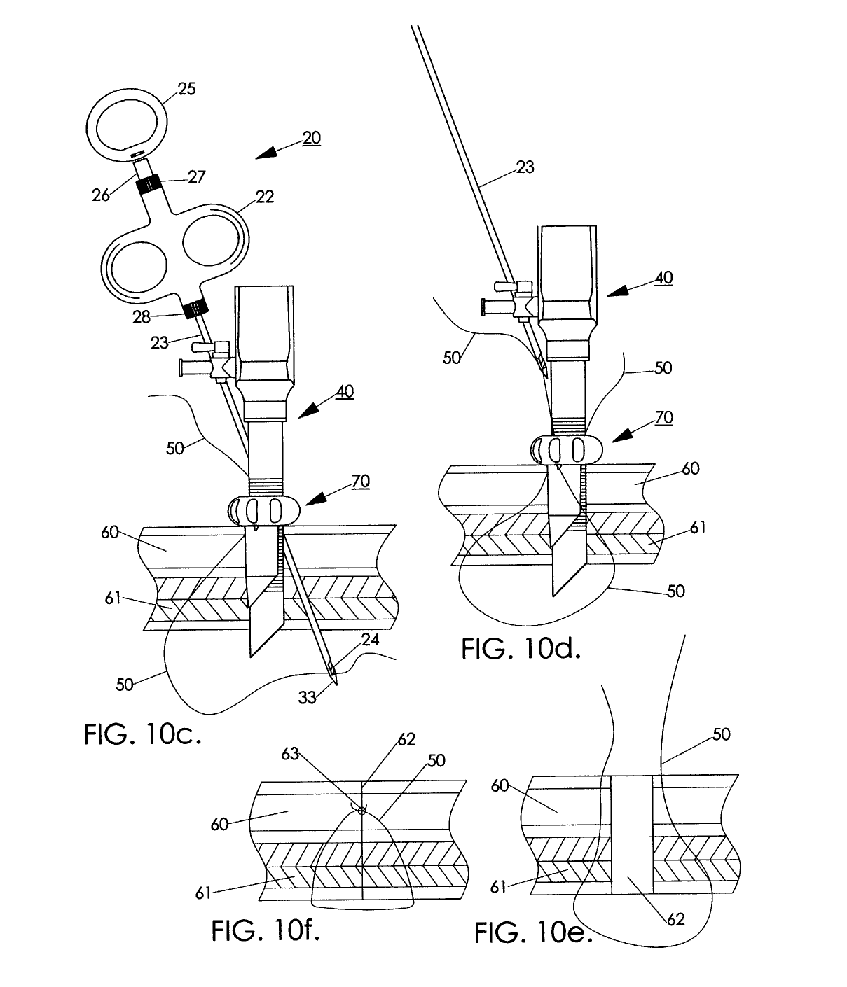

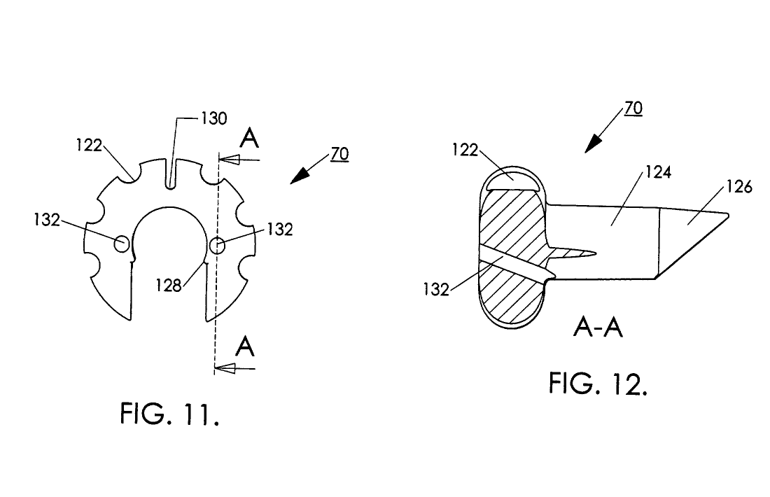

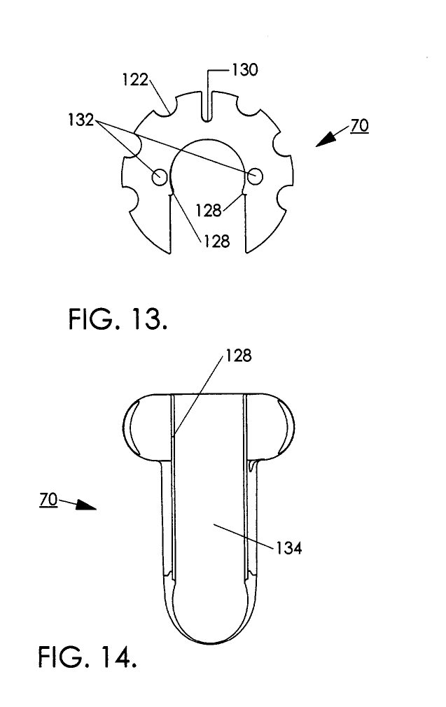

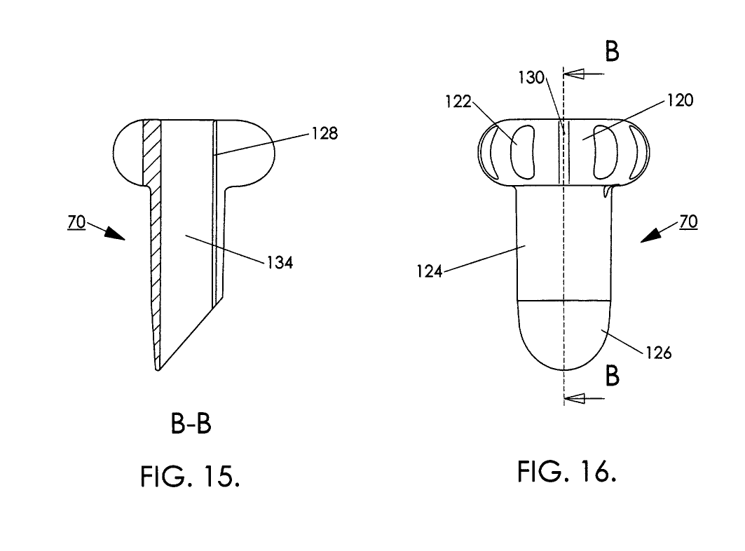

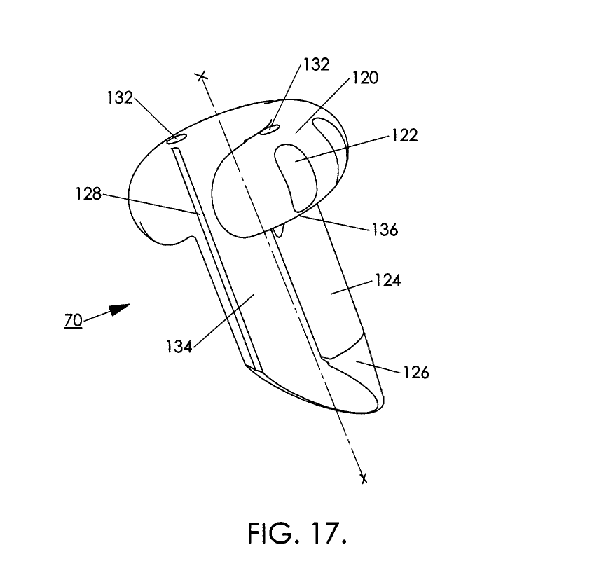

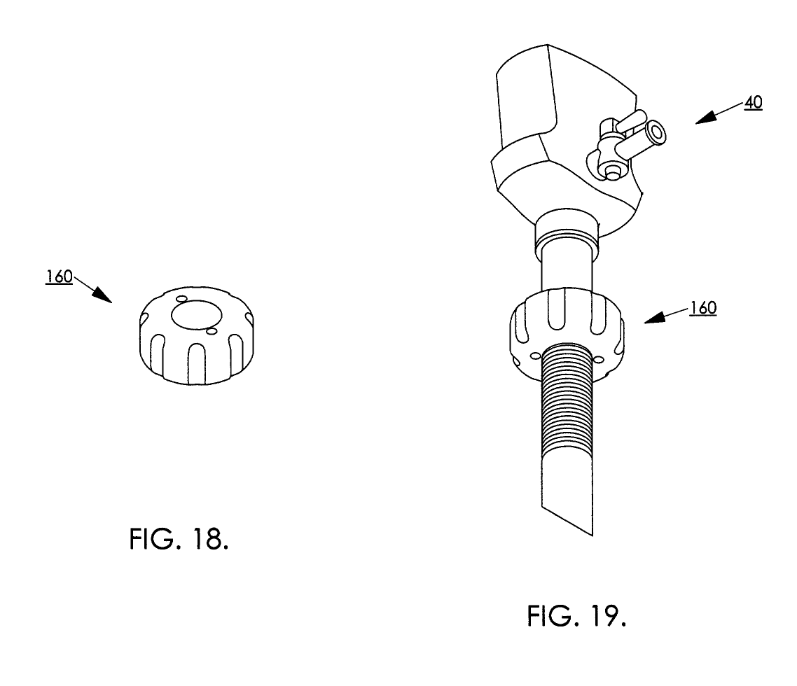

As shown in FIGS. 11-17, a specially adapted guide 70 can be used in the suturing procedure discussed above, and its application is demonstrated in FIGS. 10a-10f. The guide 70 provides the surgeon a device and methodology for accurately and precisely positioning and removing the suture material 50 in or from the patient's body where desired.

The guide 70 has a longitudinal axis x shown in FIG. 17 and is generally symmetrical about its x axis. Its extended lip also serves as a gripping area for the surgeon with a radially disposed surface 120 which further assists the surgeon in gripping and holding the guide 70. The gripping surface 120 may be smooth or roughened for easy finger contact.

The surface at the proximal end exposes the two passageways 132 through the guide 70. The passageways 132 are parallel to each other, and each forms an angle alpha of approximately 20 .degree. with the longitudinal axis x. The two passageways 132 starts at the same surface and runs in an opposite direction from each other. The diameter of the passageways 132 are sized to receive the surgical instrument to be used.

The guide 70 has an opening running parallel to the longitudinal axis x. The open side allows the surgeon to attach the guide 70 onto the trocar 40. In use, the guide 70 snaps onto the shaft of the trocar 40 still in the wound, and its extending lip 136 is adjacent to the wound to be sutured. The shaft of the trocar 40 is concentric with the center opening of the guide 70, and it clips into the center opening of the guide by two extruded locking surface 128 parallel to the longitudinal axis x. The cutout 130 in the guide 70 serves as a stress relief and makes the guide more flexible while attaching onto the trocar 40, allowing the operator to complete the snap-on attachment of the guide 70 and the trocar 40 with ease. The two extruded locking surface than keeps the guide 70 in place in order to complete skin closure. The lip 136 prevents the guide 70 from sliding further down on the trocar 40 shaft into the wound and, therefore, should be sized to be of a greater diameter than that of the open wound to be sutured.

A distal portion 126 of the guide 70 is slightly tapered . Tapering allows for greater ease of insertion while the guide 70 slides into the wound between the trocar 40 and surrounding tissue until it stops at the subcutaneous tissue by the lip portion 136.

The entire guide 70 can be integrally formed out of high-density polyethylene or other comparable material which is durable and medically inert and can serve as a disposable or a reusable product.

As can be seen in FIGS. 10a-10f, the guide 70 greatly assists in the procedure described above for FIGS. 8a-8e. More particularly, the guide 70 is attached on the trocar which is already going through the skin incision, muscle, fascia, and peritoneum so that the trocar 40 appears in the view of the laparoscope. The guide 70 is oriented so that the passageways 132 in the guide 70 are in the required position to complete skin closure.

The fascia closure instrument 20 (or 80 or 100) is inserted with suture in its grasp through the correctly positioned passageway in the guide 70 and observed to exit through the peritoneum by laparoscopic view.

The suture is then released and the instrument 20 (or 80 or 100) withdrawn from the guide 70. The instrument 20 is placed in the first passageway 132 of the guide and watched by laparoscopic view to exit through the peritoneum.

The suture is withdrawn through the hole made by the instrument 20. The trocar 40 with the guide 70 on its shaft is then withdrawn from the incision 62 completely. The suture is then tied by standard techniques, thus ensuring the fascia and peritoneum in a mass closure under the skin.

As can be seen by inspection of the Figures, particularly FIGS. 9a-9f some surgical instruments that do maintain a linear configuration could use the guide 70 with its straight passages.

Alternative embodiment to the suture guide shown in FIGS. 18-19.

The overall length of the guide shaft 124 may vary, but it does not depend upon the patient's anatomy. The guide clips onto the trocar 40 shaft which is placed in the wound at the beginning of the laparoscopic surgery. Therefore the guide shaft 124 can be eliminated completely leaving the extended lip portion 136 as an alternative guide 160 to the original suture guide 70 of the present invention as shown on FIG.18.

As shown on FIG. 19., the alternative embodiment 160 of the suture guide 70 can be placed on the shaft of the operating trocar 40 before the first surgical incision is performed assuring that when the laparoscopic surgery is complete, the suture guide will be available immediately to begin tissue closure.

Additionally, the alternative suture guide 160 can be sterilized and packaged together with the operating trocars by healthcare manufacturers, offering great economical and surgical advantages.

There has been described and illustrated herein an improved laparoscopic instrument, a guide, and a surgical method. While particular embodiments of the invention have been described, it is not intended that the invention be limited exactly thereto, as it is intended that the invention be as broad in scope as the art will permit. The foregoing description and drawings will suggest other embodiments and variations within the scope of the claims to those skilled in the art, all of which are intended to be included in the spirit of the invention as herein set forth.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is: Suture passing surgical instrument, guide and methodology for using same comprising:

1. A suture passing and grasping surgical instrument carrying suture material to an area within the body for closing an open wound, comprising:
a hollow tube having a first end and a second end;
a needle tip end and a base section, said base section fixedly engaging said first end of said hollow tube, said tip end tapering downwardly to said base section;
driving means engaging said second end of inner rod for imparting reciprocal motion to said inner rod relative to said tube for motion of said second grasping surface, wherein said second grasping surface engages said first grasping surface defining an area for grasping, carrying and passing the suture through a patient's skin for suturing an open wound and said tube, inner rod, first and second grasping surfaces and said actuating means detachable for cleaning.

2. A laparoscopic instrument for passing through a patient's peritoneum and fascia, and closing an open wound with suture material comprising:
(a) detachable piercing, carrying and grasping means having a sharp tip for insertion below skin level and grasping and carrying the suture material;
(b) driving means for driving said detachable means through the patient's peritoneum and fascia at a first point and actuating said detachable means for gripping the suture material, said detachable piercing and grasping means comprises first and second grasping surface concentricly engaged to each other, said first grasping surface fixedly engaging a hollow tube and said second grasping surface at the first end of the reciprocally moving inner part narrows down to a thin wire rod concentrically located within said hollow tube;
(c) said first and second forcep grasping surfaces having an angle, said interior grasping surfaces are paralell to each other; and
(d) said interior grasping surfaces capable for gripping various size suture while allowing the grasping surfaces to close, whereby the suture material is carried by said detachable means and recovered by driving said detachable means through the patient's peritoneum and fascia at a second point and actuating said detachable means for gripping and pulling the suture material outside the wound providing for rapid closure of the open wound.

3. A laparoscopic instrument according to claim 2, wherein said detachable piercing means comprises a sharp cone shaped tip at a distal end.

4. A laparoscopic instrument according to claim 2, wherein said detachable grasping means comprises a laparoscopic grasper defined by one fixed and one moving grasping surface.

5. A laparoscopic instrument according to claim 2, wherein said driving means comprises a housing means and a handscrew engaging to said housing means, wherein said handscrew means detachable from said housing means for cleaning prior to surgery.

6. A laparoscopic instrument for grasping, carrying and passing suture through a patient's skin for suturing an open wound comprising:
a hollow tube having a first end and a second end;
a needle tip end and a base section, said base section fixedly engaging said first end of said hollow tube, said tip end tapering downwardly to said base section;
driving means engaging said second end of inner rod for imparting reciprocal motion to said inner rod relative to said tube for motion of said second grasping surface, wherein said second grasping surface engages said first grasping surface defining an area for grasping, carrying and passing the suture through a patient's skin for suturing an open wound and said tube, inner rod, first and second grasping surfaces and said actuating means detachable for cleaning.

7. An instrument for grasping, carrying, and passing suture through a patient's tissue comprising
(a) a handle/actuator means for holding and activating the instrument from a closed to an open position back and forth; and
(b) a forcep means which opens and closes for grasping the suture and having the grasping surfaces paralell to each other in both open and closed positions, and wherein said forcep means holds the suture in a fully closed position while carrying the suture; and
(c) a sharp, cone shaped tip to be insertable through the patient's tissue to the place of suturing.

8. A device for guiding and positioning a surgical instrument carrying suture material to an area within the body for closing an open wound, comprising:
a guide means having a longitudinal axis and distal and proximal ends, and further having a first passage including a lip near said proximal end of said guide means being generally perpendicular to said longitudinal axis and a circular finger gripping surface being generally parallel to said longitudinal axis, and
said guide means defining first and second linear passageways therethrough at first and second angles generally 20 degree from said longitudinal axis, said first and second passageways terminating through said lip, said second passageway positioned oppositely adjacent said first passageway, said first and second passageways starting at a common surface; and
said guide attaches onto the shaft of the operating trocar and than it slides down on the shaft until said lip portion stops at the subcutaneous tissue above the wound to be closed, said guide positioning the surgical instrument carrying suture material for passing through said first and second passageways at said angles to the predetermined area within the body to assist in closing the open wound and the suture material can be left at a predetermined point within the body and the surgical tool may be inserted through said second passageway through said lip to retrieve the suture material from the area within the body.

9. The device of claim 8, further comprising:
an opening at the side of the said guide which is perpendicular to said longitudinal axis and said opening is used to attach said guide onto the shaft of the said trocar which remains inside the wound after a surgical procedure, therefore the said guide uses the same operating port of said trocar for optimal wound closure.

10. A method of suturing an open wound comprising the steps of:
(a)providing a surgical instrument, comprising: a sharp tip for insertion below skin level and grasping and carrying the suture material; driving means for driving said detachable means through the patient's peritoneum and fascia at a first point and actuating said detachable means for gripping the suture material, said detachable piercing and grasping means comprises first and second grasping surface concentricly engaged to each other, said first grasping surface fixedly engaging a hollow tube and said second grasping surface at the first end of the reciprocally moving inner part narrows down to a thin wire rod concentrically located within said hollow tube; said first and second forcep grasping surfaces having an angle, said interior grasping surfaces are paralell to each other; and said interior grasping surfaces capable for gripping various size suture while allowing the grasping surfaces to close completely, whereby the suture material is carried by said detachable means and recovered by driving said detachable means through the patient's peritoneum and fascia at a second point and actuating said detachable means for gripping and pulling the suture material outside the wound providing for rapid closure of the open wound;
(b) providing a guide, comprising: a longitudinal axis, and further having an opening at the side which is perpendicular to said longitudinal axis and said opening is used to attach said guide onto the shaft of the said trocar which remains inside the wound after a surgical procedure, therefore the said guide uses the same operating port of said trocar for the fascia closing procedure, and further having a first passage including a lip near said proximal end of said guide means being generally perpendicular to said longitudinal axis and a circular finger gripping surface being generally parallel to said longitudinal axis, said guide means defining therein a first generally linear passageway therethrough at a first angle less than 20 degree from said longitudinal axis, and providing for two exit holes through said lip portion above the wound to be closed, said guide positioning the surgical instrument carrying suture material for passing through said passageway at said angle to the predetermined area within the body to assist in closing the open wound;
(c) grasping the suture material between said grasping surfaces and inserting a said cone shaped tip means for passing suture material through said first passageway in said guide until reaching tissue and until said tip means is seen through the peritoneum by way of a surgical camera;
(d) releasing said suture material, withdrawing said tip of surgical instrument means from said guide, and leaving the suture material in place;
(e) inserting said tip of surgical instrument means in a second passageway defined within said guide and appositely adjacent said first passageway, said second passageway directing said tip means to a point where the suture material may first leave the body to provide wound closure, said second passageway through said guide means forming a second angle less than 20 degree from said longitudinal axis, and positioned appositely adjacent said first passageway, said first and second passageways sharing a common entrance, and further having a second exit hole through said lip, wherein the suture material can be left at the predetermined point within the body and the surgical tool may be inserted into and through said second passageway through said common entrance to retrieve the suture material from the area within the body;
(f) grasping said suture material with said grasping surfaces;
(g) retrieving said suture material with said grasping surfaces means through said guide outside the wound; and
(h) removing said surgical instrument, said guide and said trocar means from the wound and tying ends of said suture material together to provide closure of the wound.

11. The method of claim 8, further comprising: a second wing oppositely opposed to said first wing, said first and second wings defining a gap therebetween which contains a through circular opening sized as the diameter of the operating trocar in use and aligned with said first and second passageways, whereby nonlinear surgical tools may be used in conjunction with said guide.

12. A device for accurately guiding and positioning a surgical instrument carrying suture material to a predetermined area within the body for closing an open wound, comprising: a guide means having a longitudinal axis and distal and proximal ends, and further having a depth-limiting extending lip near said proximal end of said guide means and being generally perpendicular to said longitudinal axis, wherein said distal end is for insertion into the wound to a depth limited and determined by said extending lip, said guide means defining therein at least one hole providing a generally linear passageway therethrough at a first diverging angle at 20.degree. from said longitudinal axis, said passageway terminating through said lip, wherein said distal end of said guide is tapered and positionable within said trocar with the lip portion above the wound to be closed, said guide positioning the surgical instrument carrying suture material for passing through said passageway at said angle to the predetermined area within the body to assist in closing the open wound.

13. The device of claim 8 further comprising a second passageway through said guide means forming a second angle less than 20.degree. from said longitudinal axis, and positioned appositely adjacent said first passageway, said first and second passageways sharing a common surface for entering, and further said second passageway terminating through said lip, wherein the suture material can be left at the predetermined point within the body and the surgical tool may be inserted into and through said second passageway through said common entrance to retrieve the suture material from the area within the body.

14. The device of claim 9 wherein said guide means available is a plurality of sizes having different radial diameters depending on the size of the oparating trocar used for the particular surgery.

15. The device of claim 8 wherein said guide means is integrally formed of thermoplastics, approved by the medical industry.

16. A method of suturing an open wound comprising the steps of:
(a) providing a guide, comprising: a guide means having a longitudinal axis and distal and proximal ends, and further having a first passage including a lip near said proximal end of said guide means being generally perpendicular to said longitudinal axis and a circular finger gripping surface being generally parallel to said longitudinal axis, and said guide means defining first and second linear passageways therethrough at first and second angles generally 20 degree from said longitudinal axis, said first and second passageways terminating through said lip, said second passageway positioned oppositely adjacent said first passageway, said first and second passageways starting at a common surface; and said guide attaches onto the shaft of the operating trocar and than it slides down on the shaft until said lip portion stops the said guide at the subcutaneous tissue above the wound to be closed, said guide positioning the surgical instrument carrying suture material for passing through said first and second passageways at said angles to the predetermined area within the body to assist in closing the open wound and the suture material can be left at a predetermined point within the body and the surgical tool may be inserted through said second passageway through said lip to retrieve the suture material from the area within the body;
(b) providing a guide, comprising: a longitudinal axis, and further having an opening on its side which is perpendicular to said longitudinal axis and said opening is used to attach said guide onto the shaft of the said trocar which remains inside the wound after a surgical procedure, therefore the said guide uses the same operating port of said trocar for the fascia closing procedure, and further having a first passage including a lip near said proximal end of said guide means being generally perpendicular to said longitudinal axis and a circular finger gripping surface being generally parallel to said longitudinal axis, said guide means defining therein a first generally linear passageway therethrough at a first angle less than 20 degree from said longitudinal axis, and providing for two exit holes through said lip portion above the wound to be closed, said guide positioning the surgical instrument carrying suture material for passing through said passageway at said angle to the predetermined area within the body to assist in closing the open wound;
(c) grasping and inserting a tip means for passing suture material through said first passageway in said guide until reaching tissue and until said tip means is seen through the peritoneum by way of an endoscopic camera;
(d) releasing said suture material, withdrawing said tip means from said guide, and leaving the suture material in place;
(e) inserting said tip means in a second passageway defined by said hole within said guide and appositely adjacent said first passageway, said second passageway directing said tip means to a point where the suture material may first leave the body to provide wound closure;
(f) grasping said suture material with said grasping surfaces means;
(g) retrieving said suture material with said grasping surfaces means through said guide outside the wound; and
(h) removing said guide and said trocar means from the wound and tying ends of said suture material together to provide closure of the wound.
